(19) United States
(12) Patent Application Publication
(10) Pub. No.: US 2016/0169851 A1
(43) Pub. Date: Jun. 16, 2016

(54) APPARATUS AND METHOD FOR JUDGING SENSIBILITY OF SMELL

(71) Applicant: HYUNDAI MOTOR COMPANY, Seoul (KR)

(72) Inventors: Tae Hee LEE, Hwaseong-si (KR); Ki Young YOON, Seoul (KR); Ji Wan KIM, Yongin-si (KR); So Yoon PARK, Seoul (KR); Seung Chul HA, Suwon-si (KR)

(21) Appl. No.: 14/930,207

(22) Filed: Nov. 2, 2015

(30) Foreign Application Priority Data
Dec. 11, 2014 (KR) 10-2014-0178148

(51) Int. Cl. G01N 33/00

(52) U.S. Cl. CPC G01N 33/0001

(57) ABSTRACT

An electronic nose apparatus is capable of replacing the olfactory sense of human. An apparatus and method for judging sensibility of a smell form olfactory characteristics of smells as data and judge the type and intensity of the smell based on the data. Particularly, a smell pattern is implemented as a database, and the sensibility of the smell of unknown gas is judged based on the implemented data, thus providing information on the sensibility of the smell. The apparatus and method implement detecting a gas via a sensor apparatus, comparing a detection pattern detected via the sensor apparatus with a smell sensibility pattern on a previously stored database to analyze the detection pattern, determining a most approximate smell sensibility pattern, based on the comparison, and judging smell sensibility of the detection gas based on the determined smell sensibility pattern.

### CROSS-REFERENCE TO RELATED APPLICATION

This application claims under 35 U.S.C. §119(a) the benefit of priority to Korean Patent Application No. 10-2014-0178148 filed on Dec. 11, 2014, the entire contents of which are incorporated herein by reference.

### TECHNICAL FIELD

The present disclosure relates to an apparatus and method for judging sensibility of smell. More particularly, the disclosure relates to a technology for judging sensibility of smell of an unknown mixture of gas by detecting the unknown mixture and then analyzing a detected pattern of the gas.

### BACKGROUND

A conventional electronic nose is configured to detect smell molecules using an array of multiple universal sensors and then analyze the detected result in the form of a pattern, thus identifying components.

For example, Korean Patent Laid-open Publication No. 10-2003-0093682 (hereinafter referred to as ‘reference 1’) discloses a small-sized electronic nose system for detecting and identifying environmentally harmful gas and impurities contained in gasoline of a vehicle.

The electronic nose system of reference 1 is configured to have an individual sensor corresponding to respective gas in the form of an array, and to detect and analyze associated gas by processing information obtained by the sensor with an artificial neural network system.

However, reference 1 is intended to solve a problem wherein a sensor for detecting a specific gas may be influenced by other gases, so that it is difficult to optionally detect the specific gas. Thus, the electronic nose system of reference 1 merely serves to precisely detect the component of the specific gas.

Hence, the conventional electronic nose system, as described, is problematic in that it does not perform the function of a human's nose that interacts with the brain when smelling a mixture of gas and provides the sensibility of the smell of an associated gas in the mixture.

Incidentally, the smell is a type of sensibility that is very wide in an area to be expressed. Therefore, in order to judge the type of the smell, it is necessary to input data on sensibility. Since the existing electronic nose apparatus is configured to store only the gas component that is the smell component, it is impossible to store the sensibility of the smell itself.

The smell is induced by the mixing of various gas components, and may become a good smell or a bad smell due to a change in composition of the mixture of gas or a change in concentration of an individual gas within the mixture.

Therefore, assigning sensibility to the smell and categorizing the sensibility as data can find the basic cause of the smell, and may be utilized in various technical fields related to the smell.

Accordingly, there is a need for a technology that assigns sensibility to the smell and judges the sensibility, in addition to simply detecting the components of a mixture of gas.

The above information disclosed, is only for enhancement of understanding of the background of the disclosure and therefore it may contain information that does not form the prior art that is already known in this country to a person of ordinary skill in the art.

### SUMMARY

The present disclosure has been made in an effort to solve the above-described problems associated with prior art.

In one aspect, the present disclosure provides an apparatus and method for assigning sensibility of a smell and judging the sensibility of the smell when analyzing a mixture of gas.

In a preferred embodiment, a method for judging sensibility of a smell may include detecting a gas via a sensor apparatus; comparing a detection pattern detected via the sensor apparatus with a smell sensibility pattern on a previously stored database to analyze the detection pattern; determining a most approximate smell sensibility pattern, based on the comparison; and judging smell sensibility of the detection gas based on the determined smell sensibility pattern.

In another preferred embodiment, the sensor apparatus may include a sensor array including a plurality of sensors, and each of the smell sensibility pattern and the detection pattern may be a pattern based on detection values of respective sensors of the sensor array.

In still another preferred embodiment, each of the smell sensibility pattern and the detection pattern may be a pattern obtained by radially expressing the detection values of the respective sensors.

In yet another preferred embodiment, the method may further include selecting a sensor depending on an object to be detected, prior to detecting the gas.

In still yet another preferred embodiment, the smell sensibility pattern may include a plurality of pieces of pattern data that vary depending on a change in smell intensity, for each smell sensibility, and at the judging the smell sensibility, the smell intensity as well as a type of the smell sensibility may be determined.

In a further preferred embodiment, the method may further include inputting smell sensibility data to the detection pattern and storing the smell sensibility data in the database, after or during the judging the smell sensibility.

In another further preferred embodiment, the inputting and storing the smell sensibility data to the detection pattern may be performed only when it is determined that there is no previously stored data on the smell sensibility pattern which matches the detection pattern.

In still another further preferred embodiment, when it may be determined whether the detection pattern matches the previously stored data of the smell sensibility pattern, it may be determined that there is no matching if a threshold value for determining the matching is preset and data on a pattern at which a difference between a value of the previously stored data on the smell pattern and a measured value exceeds the threshold value is selected as a most approximate data.

In yet another further preferred embodiment, an apparatus for judging sensibility of a smell may include a sensor array comprising a plurality of sensors; and a control board storing data on a plurality of smell sensibility patterns and comparing the stored smell sensibility pattern with a detection pattern detected by the sensor array, thus judging sensibility of a smell of a detection gas, wherein the control board may be configured to determine a smell sensibility pattern that is most approximate to the detection pattern, based on the comparison, and judge the sensibility of the smell of the detection gas based on the determined smell sensibility pattern.

In still yet another further preferred embodiment, each of the smell sensibility pattern and the detection pattern may be a pattern comprising detection values of respective sensors of the sensor array.

In a still further preferred embodiment, each of the smell sensibility pattern and the detection pattern may be a pattern obtained by radially expressing the detection values of the respective sensors.

In a yet still further preferred embodiment, the smell sensibility pattern may include a plurality of pieces of pattern data that vary depending on a change in smell intensity, for each smell sensibility, and the control board may judge a type of the smell sensibility as well as the smell intensity.

In a yet still further preferred embodiment, the respective sensors of the sensor array may comprise sensors that may detect a main component of the smell to be detected.

In a yet still further preferred embodiment, the sensor array may be a sensor array including two or more sensors having detectability for at least one selected from a group consisting of VOCs, H₂S, NH₃, H₂, EtOH, trimethylamine, alcohol, solvent vapors, methane, CO CFC's, CO₂, O₃, and NO₂.

In a yet still further preferred embodiment, the apparatus may further include a pump supplying the detection gas to the sensor array, a manifold for evenly distributing the gas, injected from the pump, to the respective sensors of the sensor array; and a fan discharging the injected gas.

In a yet still further preferred embodiment, the apparatus may further include a display outputting data on smell sensibility judged by the control board to outside.

In a yet still further preferred embodiment, the apparatus may further include an input device inputting the smell sensibility for the detection gas.

Other aspects and preferred embodiments of the invention are discussed infra.

It is understood that the term “vehicle” or “vehicular” or other similar terms as used herein are inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

### BRIEF DESCRIPTION OF THE DRAWINGS

The above and other features of the present disclosure will now be described in detail with reference to certain exemplary embodiments thereof illustrated the accompanying drawings which are given hereinbelow by way of illustration only, and thus are not !imitative of the present disclosure, and wherein:

FIG. 1 is a flowchart schematically illustrating a method for judging sensibility of a smell, according to a preferred embodiment of the present invention,

FIGS. 2A-2C and 3A-3C illustrate smell patterns formed by smells emitted from various vehicles.

FIGS. 4A-4F is a gas-specific smell pattern analysis view.

FIGS. 5A-5B is a concentration-specific smell pattern analysis view for a specific gas.

FIG. 6 is a flowchart illustrating a method for judging sensibility of a smell, according to another preferred embodiment of the present invention.

FIG. 7 is a view illustrating the schematic configuration of an apparatus for judging sensibility of a smell, according to an embodiment of the present invention.

It should be understood that the appended drawings are not necessarily to scale, presenting a somewhat simplified representation of various preferred features illustrative of the basic principles of the disclosure. The specific design features of the present disclosure as disclosed herein, including, for example, specific dimensions, orientations, locations, and shapes will be determined in part by the particular intended application and use environment.

In the figures, reference numbers refer to the same or equivalent parts of the present disclosure throughout the several figures of the drawing.

### DETAILED DESCRIPTION

Hereinafter reference will now be made in detail to various embodiments of the present invention, examples of which are illustrated in the accompanying drawings and described below. While the disclosure will be described in conjunction with exemplary embodiments, it will be understood that present description is not intended to limit the disclosure to those exemplary embodiments. On the contrary, the disclosure is intended to cover not only the exemplary embodiments, but also various alternatives, modifications, equivalents and other embodiments, which may be included within the spirit and scope of the disclosure as defined by the appended claims.

The present disclosure aims to implement an electronic nose apparatus that is capable of replacing the olfactory sense of the human, and provides an apparatus and method for judging sensibility of a smell by forming olfactory characteristics of smells as data and judging the kind and intensity of the smell based on the data.

Particularly, the present disclosure is characterized in that a smell pattern is implemented as a database, and the sensibility of the smell of an unknown gas is judged based on the implemented data, thus providing information on the sensibility of the smell.

To this end, the present disclosure includes a sensor apparatus for judging the sensibility of the smell of the unknown gas. Such a sensor apparatus includes data about a smell pattern to identify the smell, and a learning algorithm for determining matching with the pattern data.

Particularly, in the present disclosure, the term “smell sensibility” refers to a name input into a database so as to specify sensibility of the smell that is not standardized. The term “smell sensibility pattern” refers to a pattern that expresses inherent characteristics of the smell sensibility in a predetermined manner related to a detected value of a sensor.

Further, the sensor apparatus is equipped with a sensor array including a set of sensors having different gas detectabilities. Preferably, the sensor apparatus is configured to immediately extract smell sensibility without detecting a component of a mixture of gas from the sensor array. The extracted smell sensibility may include information on the kind, intensity and other characteristics of smells.

Hereinafter, the apparatus and method for judging the smell sensibility according to a preferred embodiment of the present invention will be described in detail with reference to the accompanying drawings.

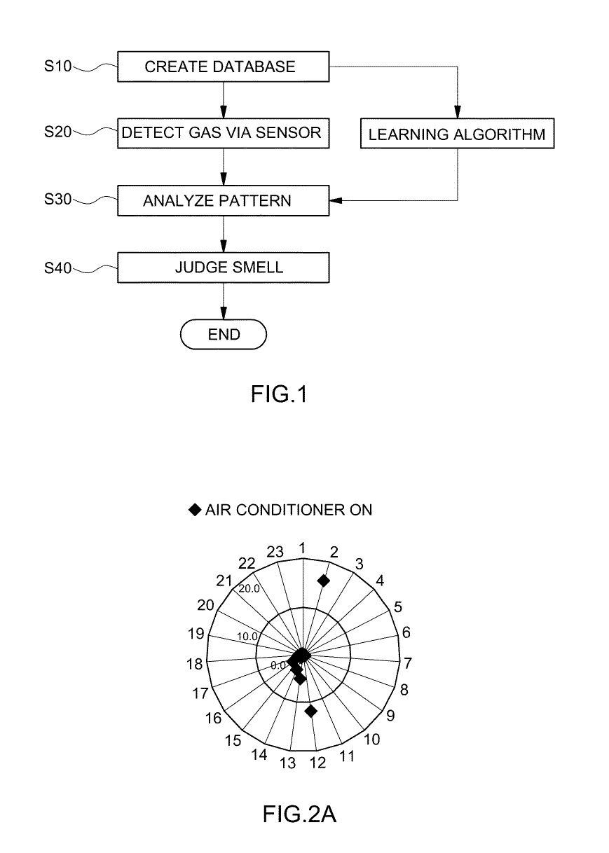

FIG. 1 is a flowchart schematically illustrating a method for judging smell sensibility, according to an embodiment of the present invention.

As shown in FIG. 1, the method for judging the smell sensibility according to the present disclosure may include a step S10 for creating a database for a smell sensibility pattern as a preliminary step. Essentially, the method may include a step S20 for detecting a gas via the sensor apparatus, a step S30 for analyzing the pattern of a detection gas, and a step S40 for judging smell sensibility.

Particularly, the step for analyzing the gas pattern is configured to compare a smell sensibility pattern on a previously stored database with a detection pattern detected via the sensor apparatus and then judge a most approximate smell sensibility pattern as the smell sensibility of the detected gas.

In this regard, the step of creating the database for the smell sensibility collects and stores data on the smell sensibility pattern stored in the sensor apparatus.

In this step, sensibility information on the smell matches with results of the smell measured by the sensor. As such a process is repeated, the database for the smell sensibility pattern is created.

For example, if a smell existing in a vehicle is perceived to be similar to a smell emitted from old books, the sensibility information on the smell is input as an “old-book smell”, and a value detected by the sensor for the gas existing in a passenger compartment of an associated vehicle is stored as data on the smell sensibility pattern.

Persons may differently feel smell sensibility. Thus, by categorizing the smell sensibility, it is possible to create the effective database for the smell sensibility patterns.

For example, the smell sensibility patterns may be categorized according to region, race, age, sex or season, and may be classified to conform to conditions for respective categories. Thereby, it is possible to generate and store independent data on smell sensibility pattern.

In case of using the data on smell sensibility pattern that is classified according to category, the step of generating and storing each data on smell sensibility pattern is performed. Prior to the step of detecting the gas and analyzing the pattern, a step of selecting at least one category among region, race, age, sex and season and setting a condition associated with the selected category may be further provided.

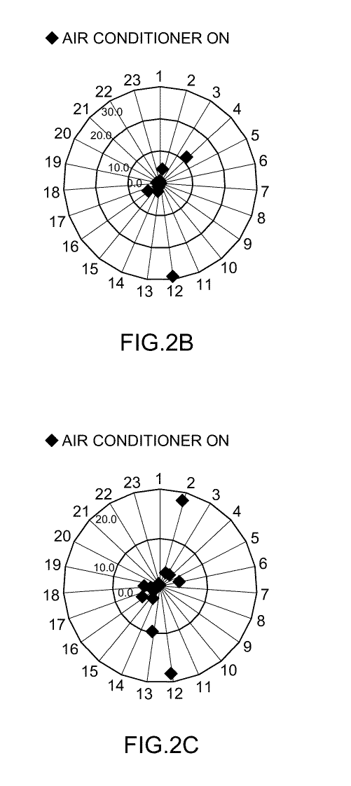

In order to aid in understanding the data on smell sensibility pattern, it will be described with reference to FIGS. 2 to 5B.

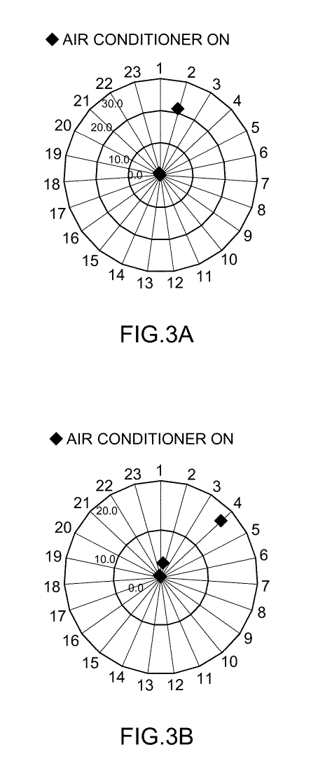

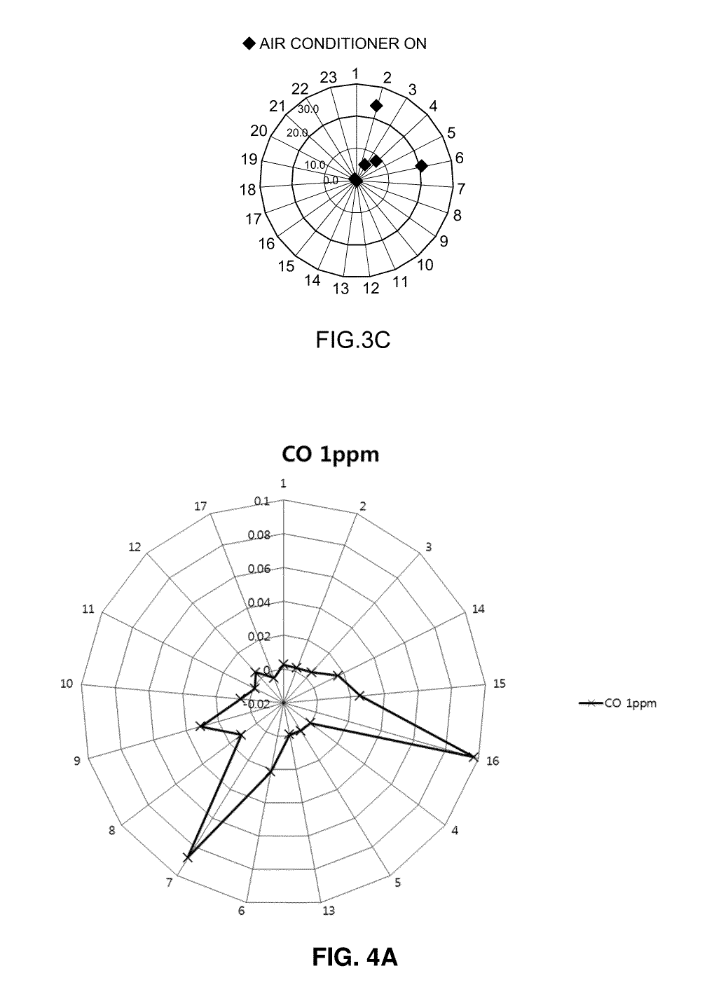

FIGS. 2 and 3 illustrate smell patterns formed by smells emitted from various vehicles.

Particularly, FIGS. 2 and 3 illustrate results of measuring gases in the vehicles via a sensor array including 23 sensors so as to measure the smells emitted from three kinds of vehicles.

The 23 sensors for the measurement are chosen to be suitable for the gases generated in the vehicles.

Table 1 shows results of detecting the smells in the vehicles by the sensor array including the 23 sensors.

TABLE 1

| | | | | Analyzed | | | | |
|---|---|---|---|---|---|---|---|---|
| | | | Minimum | Value | | Generation | Generation | |
| Type of | Serial | Composite | Detected | (Max) | Threshold | Of | Of | |
| Smell | No. | Odor | Value | ppb | Value | Gas | Smell | Formula |
| Detected | 2 | acetaldehyde | 1.5 | 76.5 | 51.0 | ◯ | ◯ | C₂H4O |
| Concentration | 4 | butylaldehyde | 0.67 | 11.8 | 17.7 | ◯ | ◯ | |
| For | 6 | valeric aldehyde | 0.41 | 6.5 | 15.9 | ◯ | ◯ | |
| Component | 11 | trimethylamine | 0.032 | 0.5 | 15.6 | ◯ | ◯ | C₃H₉N |
| [ppb] | 3 | propion aldehyde | 1.0 | 9.9 | 9.9 | ◯ | ◯ | C₃H₆O |
| | 17 | methyl | 17 | 35.1 | 2.1 | ◯ | ◯ | C₆H₁₂O |
| | | isobutylketone | | | | | | |
| | 20 | n-butyric acid | 0.19 | 0.3 | 1.6 | ◯ | ◯ | |
| | 1 | ammonia | 150 | 200.0 | 1.3 | ◯ | ◯ | H₃N |
| | 18 | butylacetate | 16 | 12.6 | 0.8 | ◯ | X | C₆H₁₂O |
| | 15 | styrene | 35 | 26.3 | 0.8 | ◯ | X | C₈H₈ |
| | 13 | m,p-xylene | 41 | 22.6 | 0.6 | ◯ | X | C₈H₁₀ |
| | 9 | dimethylsulfide | 3.0 | 1.1 | 0.4 | ◯ | X | C₂H₆O₄S |
| | 19 | propionic acid | 5.7 | 1.8 | 0.3 | ◯ | X | C₃H₆O₂ |
| | 12 | toluene | 330 | 64.3 | 0.2 | ◯ | X | C₇H₈ |
| | 16 | methylethylketone | 440 | 35.6 | 0.1 | ◯ | X | C₄H₈O |
| | 14 | o-xylene | 380 | 10.6 | 0.0 | ◯ | X | C₈H₁₀ |
| | 23 | i-butyl alcohol | 26,000 | 87.4 | 0.0 | ◯ | X | |
| | 5 | Iso valeric | 0.1 | 0.0 | 0.0 | X | X | |
| | | aldehyde | | | | | | |
| | 7 | hydrogen sulfide | 0.41 | 0.0 | 0.0 | X | X | H₂S |
| | | | | | | | | sensor |
| | 8 | methyl | 0.07 | 0.0 | 0.0 | X | X | H₂S |
| | | mercaptan | | | | | | sensor |
| | 10 | dimethyldisulfide | 2.2 | 0.0 | 0.0 | X | X | |
| | 21 | n-valeric acid | 0.037 | 0.0 | 0.0 | X | X | |
| | 22 | i-valeric acid | 0.078 | 0.0 | 0.0 | X | X | |

In the above table, the minimum detected value is a minimum value that may be sensed by a person's nose, the analyzed value is a value that is actually detected by the sensor, and the threshold value is a value that is obtained by dividing the analyzed value by the minimum detected value and represents the intensity of the smell.

Referring to table 1, the generation of gas was detected by 17 sensors and not by 6 sensors. The generation of smell was determined when at least the analyzed value of the minimum detected value was detected and the threshold value was 1 or more.

The analyzed values satisfying the above-mentioned conditions correspond to detected values by 8 sensors from above.

Pieces of information detected in three vehicles through such a measuring method are shown in FIGS. 2 and 3. Particularly, FIG. 2 indicates concentration values that are mechanical output values of the respective sensors. In the case of the vehicle shown in FIG. 2A, it can be seen that the detected value of the second sensor is the largest, and the detected value of the twelfth sensor is the second largest value.

Further, in the vehicle of FIG. 2B, it can be seen that the detected value of the twelfth sensor is the largest. In the vehicle of FIG. 2C, it can be seen that the detected value of the second sensor is similar to that of the twelfth sensor.

Meanwhile, FIG. 3 indicates the detected results of FIG. 2 as the threshold values, namely, information on smell intensity that is felt by the nose. In this case, the information related to the actual smell is the detection value of the second sensor in FIG. 3A, the detection value of the fourth sensor in FIG. 3B, and the detection values of the second and sixth sensors in FIG. 3C.

As such, the pattern including the information of the detection values on the sensor array may be used as the smell pattern that expresses a specific smell. These pieces of data on the smell pattern are accumulated, thus forming the database.

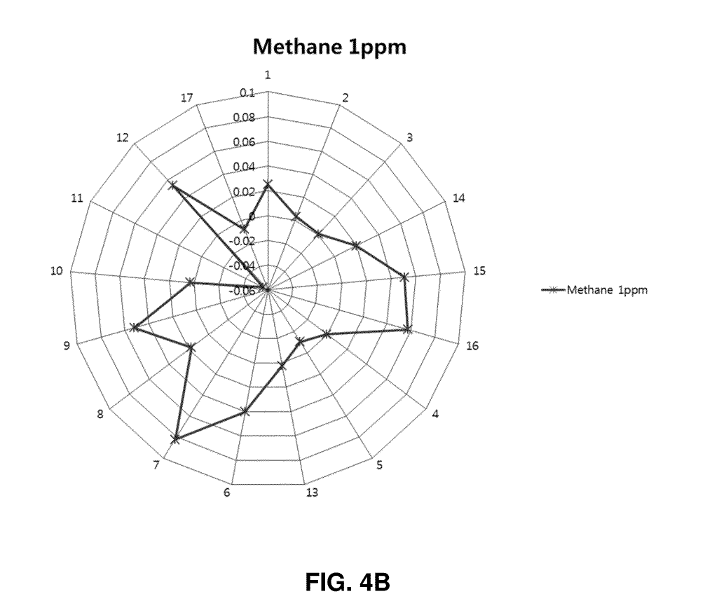

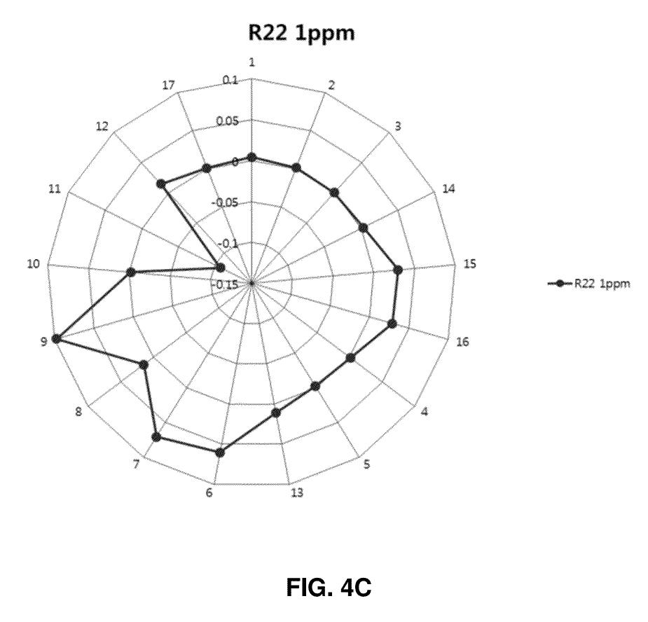

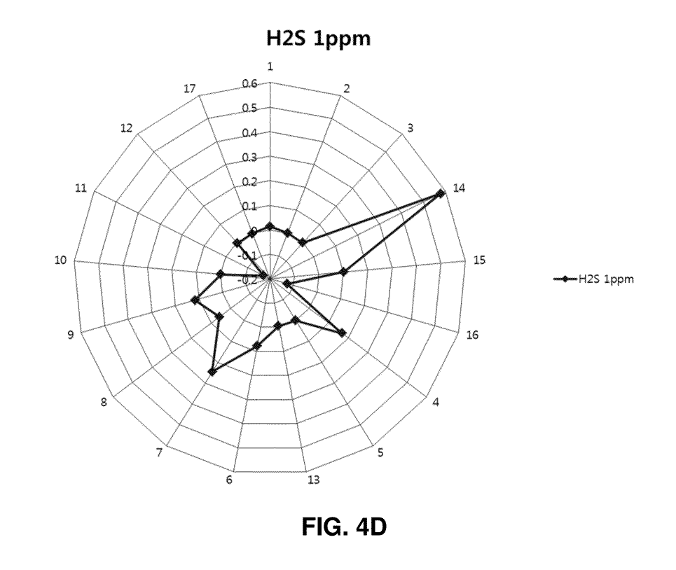

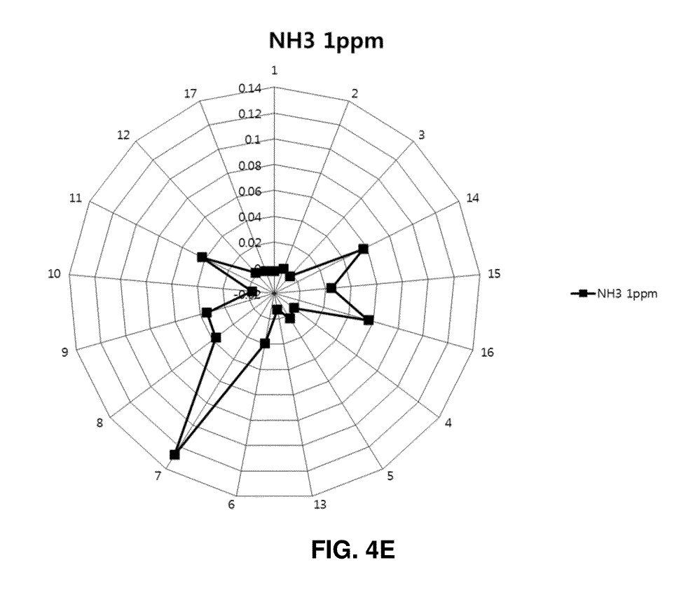

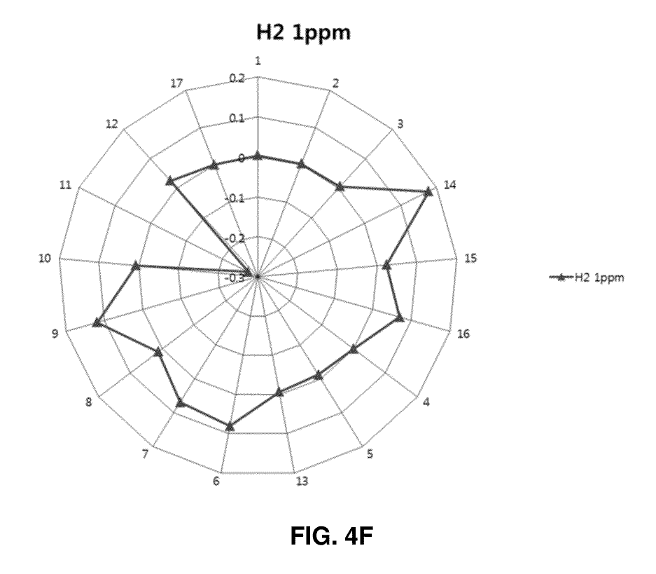

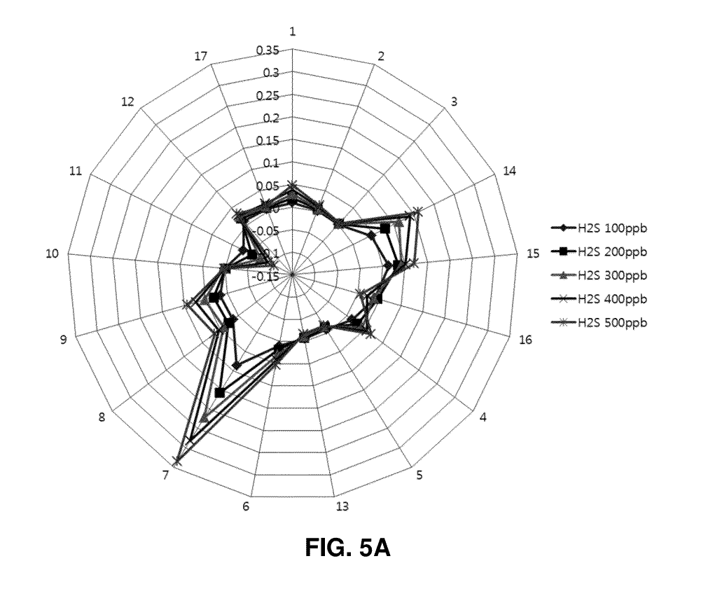

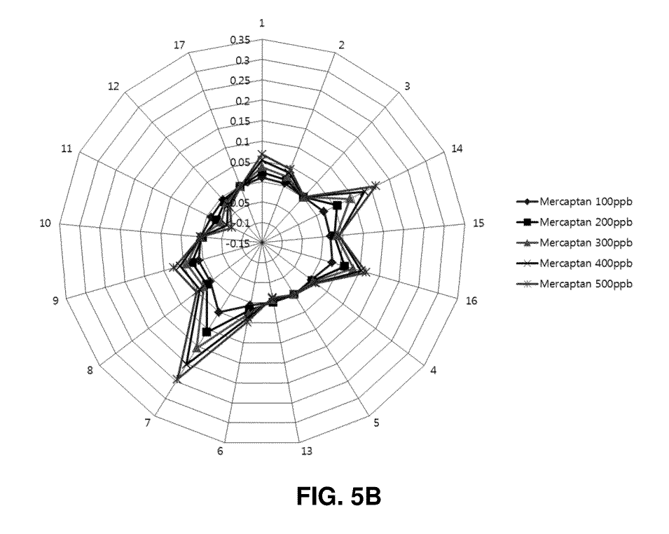

FIGS. 4A-4F and 5A-5B show a gas-specific pattern analysis view for a single gas and a concentration-specific pattern analysis view for an associated gas, respectively, as an example for indicating the tendency of the smell patterns.

FIGS. 4A-4F is a gas-specific pattern analysis view, and FIGS. 5A-5B is a concentration-specific pattern analysis view for a specific gas.

The measurement was performed by the sensor array including 17 sensors as in table 2. The selected sensor array includes sensors suitable for checking a smell in the vehicle. A step of selecting a sensor according to an object to be detected may be further provided, prior to the step of detecting the gas by the sensor apparatus.

For example, the detection process may be configured to select an optimum sensor for analyzing the pattern, rather than an output sensor showing an abnormal value among values of the respective sensors of the sensor array.

In addition, sensor verification may be performed during the selection of the sensor. In this case, by comparing data that stores an initial value of the sensor for a reference gas with currently detected data, sensor verification may be implemented to exclude sensors with low performance due to deterioration.

TABLE 2

| | Serial | | | | |
|---|---|---|---|---|---|
| Group | No. | Type | Detection Gas | Detection Range | |
| 1 | 1 | PID | VOCs | 0~50 | ppm |
| (3-CH) | 2 | PID | VOCs | 0~60 | ppm |
| | 3 | PID | H₂S | 0~100 | ppm |
| 2 | 4 | E.C. | H₂S | 0~100 | ppm |
| (2-CH) | 5 | E.C. | NH₃ | 0~100 | ppm |
| 3 | 6 | S.C. | H₂ | 1~30 | ppm |
| (7-CH) | 7 | S.C. | EtOH | 1~30 | ppm |
| | 8 | S.C. | Trimethlyamine, H₂S | 1~10 | ppm |
| | 9 | S.C. | Alchol, Solvent vapors | 50~5,000 | ppm |
| | 10 | S.C. | Methane, CO | CH₄: 500~12,500 | ppm |
| | | | | CO: 50~1,000 | ppm |
| | 11 | S.C. | CFC's | 5~100 | ppm |
| | 12 | S.C. | NH₃, H₂S | 10~300 | ppm |
| 4 | 13 | IR | CO₂ | 0~5,000 | ppm |
| 5 | 14 | S.C. | O₃ | 10~1,000 | ppb |
| (3-CH) | 15 | S.C. | NO₂ | 0.05~5 | ppm |
| | 16 | S.C. | CO/VOC | 1~1,000 | ppm |
| 6 | 17 | T&H | T/H | | |

The values detected by the respective sensors may be expressed as in the diagram of FIGS. 4A-4F. As shown in FIGS. 4A-4F, the value detected by the sensor for a single gas may be designed in the form of patterns that are obtained by combining values detected by various sensors with each other. These patterns are distinguished from each other as in FIGS. 4A-4F.

Since the gas patterns are equally applied to the mixture of gas as well, the pattern for the sensibility of the smell is created based on the database as in FIGS. 4A-4F and then is stored in the sensor apparatus.

Moreover, FIGS. 5A-5B shows an example where the pattern is analyzed according to the concentration of hydrogen sulfide and mercaptan. From the diagram of FIGS. 5A-5B, it can be seen that similar shapes of patterns are extended as the concentration of an associated gas increases.

Thus, it can be seen that the pattern for the specific smell maintains its shape regardless of an increase or reduction in concentration. That is, since the shape of the pattern is maintained, it is possible to judge the sensibility of the smell by adjusting the scale of the pattern depending on the increase or reduction in concentration.

However, in the preferred embodiment of the present invention, it is possible to draw an accurate result for the smell sensibility including the intensity of smell, by retaining data on the extended pattern of the smell sensibility including such a change in concentration.

In this case, the smell sensibility pattern includes multiple pieces of pattern data for each smell sensibility, which vary depending on a change in smell intensity. It is possible to judge the smell intensity as well as type of the smell sensibility based on the pieces of data.

Therefore, the method of judging the smell sensibility according to the preferred embodiment of the present invention compares the data of the smell sensibility patterns having the shapes of FIGS. 4A-4F and 5A-5B with the smell pattern for the actually detection gas, thus judging the sensibility for the smell.

In this case, these smell sensibility patterns are patterns composed of the detected values of the respective sensors constituting the sensor array. Preferably, they form a pattern that is obtained by radially expressing the detected values of the respective sensors, as shown in FIGS. 4A-4F.

However, the smell sensibility pattern of the present disclosure includes all shapes of patterns that may be visualized, for example, a graph of broken line, a bar graph, etc. without being limited to the radial pattern of FIGS. 4A-4F.

In other words, any shape is possible as long as inherent data forming the smell sensibility pattern can be visually expressed.

In this connection, the sensors for detecting the gas respond to components other than a target gas component. Such a response result is reflected on the detected value.

Particularly, according to the present disclosure, a single sensor for detecting a specific gas or smell is not individually used, but a set of sensors each having detectability for the specific gas is used. The detected values of the sensors are collectively patterned and used.

That is, the respective sensors output results responding to all components of the unknown mixture of gas. Based on the collectively detected results, the pattern is determined. Thus, if the smell sensibility for the unknown gas is determined, the determined pattern for the smell sensibility becomes an overall pattern by the detection values of the plurality of sensors. Similarly, the pattern for the gas to be measured is also determined by the overall detection values of the set of sensors in the same manner.

Incidentally, at the step of analyzing the smell pattern, the smell pattern on the created database is compared with the pattern detected by the sensor array, thus determining an approximate pattern.

In this regard, it is possible to use a predetermined learning algorithm. Such a learning algorithm is implemented as an algorithm that may verify the similarity of the patterns shown in FIGS. 4A-4F. As shown in FIGS. 1 and 4A-4F, the learning algorithm can receive data from the database of smell sensibility patterns, created at step S10, verify similarity of the patterns and provide the verification results for the analysis of step S30.

Thus, the smell sensibility for the detection gas is judged from the most approximate pattern on the database, at step S40 of FIG. 1.

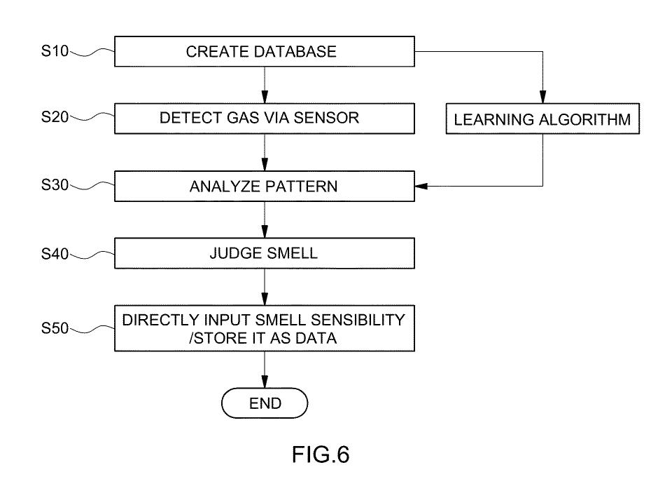

FIG. 6 is a flowchart illustrating a method for judging smell sensibility according to another preferred embodiment of the present invention.

The embodiment of FIG. 6 further includes a step of directly inputting smell sensibility for a detected detection gas. That is, this embodiment is configured to perform the steps S10 to S40 in the same manner as the preceding embodiment, as discussed with regards to FIG. 1. However, after the smell judging step S40, the embodiment of FIG. 6 is configured to directly input the smell sensibility felt by an experimenter after he or she directly smells the gas, separately from or simultaneously with the smell judging step, at step S50.

That is, at step S50, a user directly inputs the smell sensibility for the detection gas, and then it is stored in the form of data. Preferably, the data for the input smell sensibility forms new data on a smell sensibility pattern along with the data on the detection pattern. This data constitutes a part of database for the smell sensibility pattern.

In this connection, the step S50 is preferably performed only when it is determined in a data-matching checking operation that detected data on smell sensibility pattern is slightly different from previously stored data on smell sensibility pattern.

In this case, at the smell sensibility judging step S40, the most approximate pattern is determined as described above. The matching between pieces of data is determined by comparing a numerical value of each pattern with a measured value, digitizing a difference between the numerical value and the measured value, and then comparing the difference with a threshold value for determining the matching.

For example, if the threshold value for determining the matching is preset and data of a pattern at which the difference between the value of previously stored data on the smell pattern and the measured value exceeds the threshold value is selected as the most approximate data, it may be determined that there is no matching data among the previously stored pieces of data on pattern.

In this case, according to this embodiment, it is preferable that the mismatching of data be indicated and a user directly input the data on smell sensibility of an associated gas.

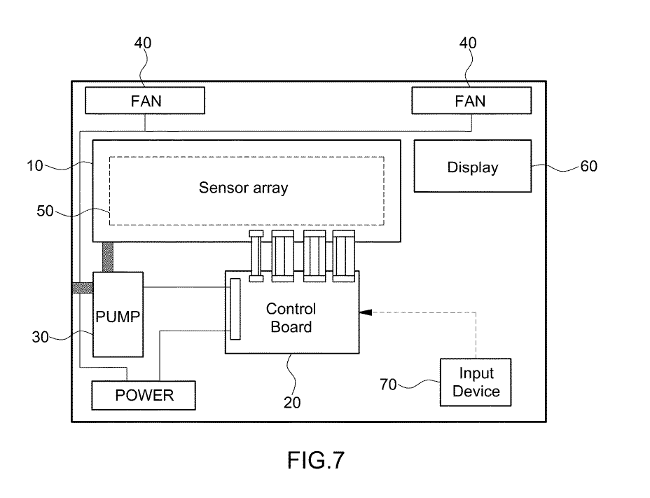

Furthermore, FIG. 7 illustrates the schematic configuration of an apparatus for judging smell sensibility to implement the above-mentioned method for judging the smell sensibility.

Referring to FIG. 7, the apparatus for judging the smell sensibility according to the present disclosure includes a sensor array 10 composed of a set of sensors, and a control board 20 judging the smell sensibility from data detected by the sensor array 10.

Further, the apparatus for judging the smell sensibility according to this embodiment includes a pump 30 supplying the detection gas to the sensor array 10, a manifold 50 provided on the sensor array 10 to evenly distribute the gas, injected from the pump, to the respective sensors of the sensor array 10, and a fan 40 discharging the injected gas.

The apparatus further includes a display 60 that outputs information judged by the control board 20 to an outside, and a power source that supplies power to respective part including the control board 20.

Particularly, the respective sensors of the sensor array 10 comprise sensors that may detect main components of the smell to be detected, as described above.

By way of example, such a sensor array 10 may include 17 sensors including temperature and humidity sensors shown in table 2, and may apply proper sensors depending on the smell to be detected. For example, the sensor array 10 may include two or more sensors having detectability for at least one selected from a group consisting of VOCs, H₂S, NH₃, H₂, EtOH, trimethylamine, alcohol, solvent vapors, methane, CO CFC's, CO₂, O₃, and NO₂.

Thus, the sensor array 10 collects output values of the respective sensors to form a detection pattern for an object to be detected.

Further, the control board 20 is configured to store data on the plurality of smell sensibility patterns and to compare the stored smell sensibility pattern with the detection pattern detected by the sensor array 10, thus judging the smell sensibility of the detection gas.

That is, after data output from each sensor of the sensor array 10 is input as the detection pattern into the control board 20, it compares the input detection pattern with various stored smell sensibility patterns, thus determining the most approximate smell sensibility pattern. Since the determined smell sensibility pattern is the smell sensibility of the detection gas, the control board 20 judges the smell sensibility of the detection gas based on the determined data and then outputs it through the display 60.

Further, as described above, according to an embodiment of the present invention, the apparatus may further include an input device 70 that is used to directly input the smell sensibility for the detection gas to be measured.

The data on the smell sensibility that is input via the input device 70 forms new data on the smell sensibility pattern along with the data measured via the sensor array. This new data is stored in the control board 20 to constitute the database.

As described above, the apparatus and method according to the present disclosure may store smell sensibility and in practice apply it to various technical fields not by detecting specific gas components contained in an unknown mixture of gas, but by assigning smell sensibility to the mixture of gas itself and sorting the sensibility based on quantified data.

Further, the apparatus and method according to the present disclosure may realize a smell judging model that is capable of completely substituting for the human sense of smell.

Furthermore, the apparatus and method for judging sensibility of a smell according to the present disclosure are utilized to perceive a smell generated in a vehicle. Therefore, the disclosure is useful for finding the cause of various types of smell generated in the vehicle and the measures of removing the smells.

The disclosure has been described in detail with reference to preferred embodiments thereof. However, it will be appreciated by those skilled in the art that changes may be made in these embodiments without departing from the principles and spirit of the disclosure, the scope of which is defined in the appended claims and their equivalents.

What is claimed is:

1. A method for judging sensibility of a smell comprising:
detecting a gas via a sensor apparatus;
comparing a detection pattern detected via the sensor apparatus with a smell sensibility pattern on a previously stored database to analyze the detection pattern;
determining a most approximate smell sensibility pattern, based on the comparison; and
judging smell sensibility of the detection gas based on the determined smell sensibility pattern.

2. The method of claim 1, wherein the sensor apparatus comprises a sensor array including a plurality of sensors, and each of the smell sensibility pattern and the detection pattern is a pattern based on detection values of respective sensors of the sensor array.

3. The method of claim 2, wherein each of the smell sensibility pattern and the detection pattern is a pattern obtained by radially expressing the detection values of the respective sensors.

4. The method of claim 1, further comprising:
selecting a sensor depending on an object to be detected, prior to detecting the gas.

5. The method of claim 1, wherein the smell sensibility pattern comprises a plurality of pieces of pattern data that vary depending on a change in smell intensity, for each smell sensibility, and wherein
at the judging the smell sensibility, the smell intensity as well as a type of the smell sensibility is determined.

6. The method of claim 1, further comprising:
inputting smell sensibility data to the detection pattern and storing the smell sensibility data in the database, after or during the judging the smell sensibility.

7. The method of claim 6, wherein the inputting and storing the smell sensibility data to the detection pattern is performed only when it is determined that there is no previously stored data on the smell sensibility pattern which matches the detection pattern.

8. The method of claim 7, wherein, when it is determined whether the detection pattern matches the previously stored data of the smell sensibility pattern, it is determined that there is no matching if a threshold value for determining the matching is preset and data on a pattern at which a difference between a value of the previously stored data on the smell pattern and a measured value exceeds the threshold value is selected as a most approximate data.

9. An apparatus for judging sensibility of a smell comprising:
a sensor array comprising a plurality of sensors; and
a control board storing data on a plurality of smell sensibility patterns, and comparing the stored smell sensibility pattern with a detection pattern detected by the sensor array, thus judging sensibility of a smell of a detection gas,
wherein the control board is configured to determine a smell sensibility pattern that is most approximate to the detection pattern, based on the comparison, and judge the sensibility of the smell of the detection gas based on the determined smell sensibility pattern.

10. The apparatus of claim 9, wherein each of the smell sensibility pattern and the detection pattern is a pattern comprising detection values of respective sensors of the sensor array.

11. The apparatus of claim 10, wherein each of the smell sensibility pattern and the detection pattern is a pattern obtained by radially expressing the detection values of the respective sensors.

12. The apparatus of claim 9, wherein the smell sensibility pattern comprises a plurality of pieces of pattern data that vary depending on a change in smell intensity, for each smell sensibility, and wherein
the control board judges a type of the smell sensibility as well as the smell intensity.

13. The apparatus of claim 9, wherein the respective sensors of the sensor array comprise sensors that may detect a main component of the smell to be detected.

14. The apparatus of claim 13, wherein the sensor array is a sensor array comprising two or more sensors having detectability for at least one selected from a group consisting of VOCs, H₂S, NH₃, H₂, EtOH, trimethylamine, alcohol, solvent vapors, methane, CO CFC's, CO₂, O₃, and NO₂.

15. The apparatus of claim 9, further comprising:
a pump supplying the detection gas to the sensor array,
a manifold for evenly distributing the gas, injected from the pump, to the respective sensors of the sensor array; and
a fan discharging the injected gas.

16. The apparatus of claim 9, further comprising:
a display outputting data on smell sensibility judged by the control board to outside.

17. The apparatus of claim 9, further comprising:
an input device inputting the smell sensibility for the detection gas.
